(19) United States
(12) Patent Application Publication
(10) Pub. No.: US 2019/0257724 A1
(43) Pub. Date: Aug. 22, 2019

(54) Functional Targeted Brain Endoskeletonization

(71) Applicant: The Board of Trustees of the Leland Stanford Junior University, Stanford, CA (US)

(72) Inventors: Karl A. Deisseroth, Stanford, CA (US); Viviana Gradinaru, La Canada Flintridge, CA (US)

(21) Appl. No.: 16/151,057

(22) Filed: Oct. 3, 2018

Related U.S. Application Data
(63) Continuation of application No. 15/090,374, filed on Apr. 4, 2016, now abandoned.
Application No. 15/090,374 is a continuation of application No. 13/980,842, filed on Oct. 1, 2013, now abandoned.
(60) Provisional application No. 61/462,131, filed on Jan. 28, 2011.

(51) Int. Cl. G01N 1/30; G01N 1/36; A61K 38/17; A61K 48/00

(52) U.S. Cl. CPC G01N 1/30; A61K 48/00; A61K 38/1748; G01N 1/36

(57) ABSTRACT

Compositions and methods are provided for TEMPEST (Target-Element Modification by Physical and Enduring Structural Transmutation), a method for creating durable structures in vivo in a cell-type and/or circuit specific manner via the use of insoluble polymers. TEMPEST provides a way to functionally remove cells while preserving their “shadow” for easy post-experiment detection and classification. The method of the invention are of particular interest for modifying neurons, which may be central nervous system or peripheral nervous system cells, however the approach may be applied to other cellular systems as well, either in culture system models or in animals.

### BACKGROUND

Understanding the circuit-level functional organization of the brain has important implications for both basic and clinical neuroscience. It has previously been shown that optical manipulation of activity in neural circuits with light-sensitive rhodopsins can help in illuminating both the normal circuit function and major disease mechanisms (see, for example, Zhang et al. (2010) Nat. Protocols 5:439). To complement the functional control capabilities of optogenetics, methods of preserving the structural integrity of defined brain circuits in vivo and in vitro are of interest.

### SUMMARY OF THE INVENTION

Compositions and methods are provided for TEMPEST (Target-Element Modification by Physical and Enduring Structural Transmutation), a method for creating durable structures in vivo in a cell-type and/or circuit specific manner via the use of insoluble polymers. TEMPEST provides a way to functionally remove cells while preserving their “shadow” for easy post-experiment detection and classification. The method of the invention are of particular interest for modifying neurons, which may be central nervous system or peripheral nervous system cells, however the approach may be applied to other cellular systems as well, either in culture system models or in animals.

In the methods of the invention, a cell, e.g. a neuron, is targeted to express a genetic sequence that directly or indirectly gives rise to a stable endoskeleton structure within the cell. TEMPEST sequences of interest may encode polymers suitable for endoskeleton structure, e.g. keratins, silks, microtubules, microfilaments, and the like; or may encode enzymes that catalyze formation of an endoskeleton from monomers normally present or provided to the cell. Cells may be targeted genetically, topologically, virally, by structure, connectivity, promoters, tropisms, or other means. The targeted cells can then form a structurally coherent and sound network. This process may be carried out with multiple endoskeletons in the same tissue. For example, any number of “split” and multicomponent strategies for crosslinking, polymerization, and durabilization may find use, including the split XFPs, split inteins, keratin-associated proteins, and the like.

Following expression of the TEMPEST sequence and deposition of an endoskeleton, the activity of the remaining non-modified cells may be studied for function, gene expression, behavior, electrochemistry, and the like, to determine the effect of selective inactivation of the targeted cells. In some embodiments, candidate agents or treatments are applied to the organ before, during or after endoskeleton deposition to determine the effect of the treatment or agent on cells in the absence or presence of the targeted cells.

The targeted cells, including the endoskeleton structure, may be modified or functionalized to provide a role of interest, including without limitation conduction of charge, conduction of drugs or fluids, conduction of growth factors or other elements, and the like. Functionalization of the durable structures allows the construction of artificial neuronal networks based on real brain connectivity with the appropriate addition of switches, modulators, etc., which may further be connected to appropriate mechanical and/or electrical circuits of interest.

The organ structure may be digested away from the targeted endoskeleton structures, e.g. to determine circuitry connections, visualization of structures, and the like, using any convenient method, e.g. hypotonic shock, enzyme digestion, heat, and the like. In order to provide additional three-dimensional support the endoskeleton cells may be embedded in any suitable matrix, e.g. collagen, resins, water, gels, foam, hydrogel, and the like.

Following endoskeleton deposition, the organ, e.g. brain, structure may be modified for various purposes. In some embodiments, specific cells of interest are detectably labeled, where the labeled cells may be the or different from the endoskeleton forming cells. Various detectable markers may be used, as known in the art, including markers that selectively bind to a cellular component, e.g. antibodies or other suitable binding partners may be used that selectively bind to the endoskeleton, or to cell surface proteins present on cells of interest, where the binding partner may be labeled with a fluorescent moiety, bioluminescent moiety, reflective moieties, conductive moieties, light-absorbing moieties, metals, and the like, in order to allow visualization and study of the endoskeleton structure. Cells may be labeled before, during, or following the endoskeleton deposition.

In some embodiments of the invention, a soluble entity is delivered to the organ of interest in a targeted manner, followed by global delivery of a durabilizing factor that acts on the soluble entity. In an alternative embodiment, the durabilizing factor is expressed in a targeted manner, followed by global delivery of a nondurable entity that interacts with the targeted cells and/or endoskeleton.

### BRIEF DESCRIPTION OF THE DRAWINGS

FIG. 1: A. Polymers optimized to fill and durabilize neurons in cell-type specific fashion. B. Keratin filaments in transfected neurons. C. Keratin but not mCherry resistant to hypotonic lysis Hair-like filaments in genetically defined neurons with viral or transgenic approaches.

FIG. 2: A. 3D neural culture in collagen in vitro 3D Enduring Networks. B. After hypotonic shock, keratinized neurons remain intact while EYFP only neurons degraded C. Cell-type specific expression in vivo. D. Multiple Networks: Cortical/Dentate Parvalbumin Inhibitory (OK8/18) and CaMKIIα Excitatory (OK85/35) neurons.

FIG. 3. chitin synthase expression in primary hippocampal cultures.

FIG. 4: different keratin pairs tested.

FIG. 5. Antibody Stain/Gold-coated neurons.

### DETAILED DESCRIPTION OF THE EMBODIMENTS

Methods and compositions are provided to generate a stable intact cell type-specific physical structure derived from intact cellular circuits. The physical structure, once deposited, can be studied for its physical connectivity, mapped functionally with regard to dynamics and circuit flow, and serve as both a source of fundamental insight into cellular circuit function, a means of mapping and understanding circuit pathologies, a technique for screening and identifying interventions to correct circuit abnormalities, a means of permanently storing or immortalizing cellular circuits in terms of structure, connectivity, identity and functionality, and a technique for extending or expanding brain function, human or otherwise, in terms of capacity, complexity, consciousness, or power.

The stable structure, or endoskeleton, may be composed of any number of encodable polymers, polymerizeable components, e.g. photopolymerizeable components, microtubules, filaments, polysaccharides, amino acids, or other polymers than can be constructed from native or non-native monomers or enzymes. For example, chitin synthetases may be used to catalyze the construction of chitin from native monomers. Alternatively keratin pairs may be expressed to provide for a keratinized endoskeleton structure.

Following endoskeleton deposition, the structure may be tagged or labeled for novel properties like electrical conductivity, e.g. by coating with conductive elements including metals, nanotubes, and the like. Multiple different classes of networks maybe created with different transduced genes or monomers or enzymes.

The connections between the endoskeletonized cells may be functionalized by any number of means. Antibodies to cap or tail of the polymeric filaments can carry conductive beads, transistors, logic elements, linkers, or gating elements that may be controlled, externally, or internally. With different classes of labeled networks, distinct interfaces targeted to different functions and roles in linking different targeted circuits or cells may be implemented with custom diverse switches, including local phosphorylation states, surface or subcellular localization, synapse size, protein concentration, or other marker of synapse gain or function to mimic local information storage and capture local memory.

Targeting may occur by various mechanisms and arrangements as noted above, including but not limited to promoters, viruses, topological targeting e.g. with retrograde transduction of transsynaptic mechanisms like WGA and TTC), or other items.

Interfaces to electronics or biologics may be implemented, and a functional, durable, immortalized and tractable circuit or brain may result.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

TEMPEST coding sequence. As used herein, the term a TEMPEST coding sequence refers to an encoded genetic entity that directly or directly gives rise to a durable structure upon expression. The resulting tough, durable endoskeleton will preserve the form of the interconnected neural circuitry. Those sequences that give rise directly to durable structures include, without limitation, encodable polymers, e.g. microtubules, filaments, keratins, silk, and the like. Nucleic acids themselves, e.g. RNA, may also give rise directly to a durable structure. Those sequences that give rise to durable structures indirectly include, without limitation, enzymes involved in the polymerization of monomers, such as polysaccharides and other native or non-native monomers. For example, chitin synthetases may be used to catalyze the construction of chitin from native monomers.

In some embodiments of the invention, the TEMPEST coding sequence encodes intermediate filaments, which includes, without limitation, a functional pair of keratin proteins. Intermediate filaments (IFs) are a structurally related family of cellular proteins that are intimately involved with the cytoskeleton. The common structural motif shared by all IFs is a central alpha-helical ‘rod domain’ flanked by variable N- and C-terminal domains. The rod domain, the canonical feature of IFs, has been highly conserved during evolution. The variable terminals, however, have allowed the known IFs to be classified into 6 distinct types by virtue of their differing amino acid sequences. Keratins compose types I and II IFs. Type I and type II keratins are usually expressed as preferential pairs, in equal proportions in cells, of type I and type II keratins. Any one of the many keratin pairs may be utilized. Exemplary pairing of keratins include, without limitation, KRT1 or KRT2 with KRT9 or KRT10; KRT3 and KRT12; KRT4 and KRT13; KRT5 and KRT14 or KRT15; KRT6 and KRT 16 or KRT17; KRT8 and KRT18 or KRT20; etc., as known in the art. Pairs commonly comprise one basic member and one acidic member.

Cells may be targeted for expression of a TEMPEST sequence genetically, topologically, virally, by structure, connectivity, promoters, tropisms, or other means. TEMPEST sequences can be selectively expressed in defined subsets of neurons in the brain using a variety of expression targeting strategies.

Viral expression systems. Viral vectors based on lentivirus and adeno-associated virus (AAV) can be used to target TEMPEST gene expression in a wide range of experimental subjects ranging from rodents to primates. Specifically, high titer lentivirus and AAV-based vectors can be easily produced in tissue-culture, or obtained through a number of virus production facilities. These transduction methods have been shown to achieve high levels of functional gene expression in neurons for several months.

Although most common AAV and lentivirus vectors carry strong ubiquitous or pan-neuronal promoters, some more specific promoter fragments retain cell type-specificity, allowing selective targeting in animals where transgenic technology is not accessible. In addition, viruses are capable of mediating high levels of gene expression by introducing multiple gene copies into each target cell, an important function for overcoming the low transcriptional activity of some cell-specific promoters. In general for rodent brains, gene expression reaches functional levels within 3 weeks after AAV injection and within 2 weeks after lentivirus injection. To reach the high steady-state levels of expression in distal axonal processes, longer periods of expression (>6 weeks) may be necessary.

Electroporation: specific cell types can also be targeted developmentally with in utero electroporation, e.g. at precisely timed embryonic days in mouse to target cortical layers II and III (E15.5), layer IV (E13.5) or layers V and VI (E12.5). In utero electroporation also can be used to express genes in the inhibitory neurons of the striatum or in the hippocampus. In addition, unlike viral delivery methods, in utero electroporation can be used to deliver DNA of any size, therefore permitting the use of larger promoter segments to achieve higher cell-type specificity. Electroporation also allows high copy number of genes to be introduced into the target cells.

Transgenic mice: transgenic technologies can be used to restrict gene expression to specific subsets of neurons in mice or rats. Using either short transgene cassettes carrying recombinant promoters or bacterial artificial chromosomes (BACs)-based transgenic constructs, TEMPEST genes can be functionally expressed in subsets of neurons in intact circuits.

Conditional expression systems: although cell-specific promoters are effective at restricting gene expression to subsets of genetically defined neurons, some promoters have weak transcriptional activity. To amplify the transcriptional activity in a cell-specific manner, conditional AAV vectors have been developed to capitalize on the numerous cell-specific Cre-driver transgenic mouse lines. These conditional AAV expression vectors carry transgene cassettes that are activated only in the presence of Cre, and the use of strong ubiquitous promoters to drive the Cre-activated transgene selectively amplifies gene expression level only in the cells of interest.

Circuit-specific cell targeting based on neuronal projection patterns: neurons identified by a given genetic marker can still be quite diverse, either receiving innervations from or sending axonal projections to distinct brain regions. For example, some of the tyrosine hydroxylase-expressing dopaminergic (DA) neurons in the midbrain innervate reward-related brain structures such as the nucleus accumbens, whereas other DA neurons project to motor control centers such as the striatum, and spatial separation between different DA neuron populations is not complete. It may be possible to selectively control a connection-defined neural pathway through focal injection of viral vectors followed by stimulation of axon terminals in the target downstream brain structure.

A number of plant and microbial proteins and several viral vectors with unique anterograde- or retrograde-transporting properties may be engineered with recombinases to activate gene expression in sub-populations of neurons with cell type- and circuit specificity. For example, expression of fusion proteins containing Cre and either wheat germ agglutinin or tetanus toxin fragment C in the cell bodies of one brain region will allow the recombinase to be trans-neuronally delivered to up- or down-stream neurons in another brain region. Similarly, viral vectors, such as rabies virus or herpes simplex virus 1 (HSV-1) vectors, can be used for retrograde gene delivery, and the H129 strain of HSV might be developed for anterograde gene delivery. When combined with conditional expression systems, either Cre-dependent transgenic mice or viral vectors, this strategy allows circuit-specific gene expression in a variety of mammalian animal models not limited to mice. Moreover, microbial protein expression can also be restricted to specific intracellular compartments and locations by fusing to targeting motifs and protein domains.

Transgenic mice: transgenic technologies can be used to restrict gene expression to specific subsets of neurons in mice or rats. Using either short transgene cassettes carrying recombinant promoters or bacterial artificial chromosomes (BACs)-based transgenic constructs, TEMPEST genes can be functionally expressed in subsets of neurons in intact circuits.

The genetic construct may be introduced into tissues or host cells by any number of routes, including calcium phosphate transfection, viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into cells.

A number of selection systems may be used for introducing the genetic changes, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk.sup.-, hgprt.sup.- or aprt.sup.-cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

By “comprising” it is meant that the recited elements are required in the composition/method/kit, but other elements may be included to form the composition/method/kit etc. within the scope of the claim. By “consisting essentially of”, it is meant a limitation of the scope of composition or method described to the specified materials or steps that do not materially affect the basic and novel characteristic(s) of the subject invention. By “consisting of”, it is meant the exclusion from the composition, method, or kit of any element, step, or ingredient not specified in the claim.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The term “gene” is well understood in the art and includes polynucleotides encoding a polypeptide. In addition to the polypeptide coding regions, a gene may include non-coding regions including, but not limited to, introns, transcribed but untranslated segments, and regulatory elements upstream and downstream of the coding segments.

The terms “polypeptide”, “peptide” and “protein” are used interchangeably to refer to polymers of amino acids of any length. These terms also include proteins that are post-translationally modified through reactions that include glycosylation, acetylation and phosphorylation.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

An “effective amount” is an amount sufficient to effect desired results. An effective amount can be administered in one or more administrations.

An “individual” is a vertebrate, preferably a mammal. Mammals include, but are not limited to, rodents, primates, farm animals, sport animals, and pets.

In other embodiments, modulation of an effect on a targeted organ is tested, where the organ is modulated before, during or after targeted endoskeleton deposition. Candidate modulatory effects include electrical stimulation, including ion alteration; administration of candidate agents; altering physiological parameters such as immune responses; introduction of cells, including without limitation stem cells such as neural stem cells; and may also include behavioral studies, such as memory, language acquisition, etc. Such screening may be performed using an in vitro model or an animal model, in which targeted cells in the model are targeted for endoskeleton deposition before or after administration. The effect of the treatment may be assessed by measuring any parameter of interest, including circuitry of the targeted neurons, behavior of non-targeted neurons, learning and cognitive function, and the like.

The term “agent” as used herein describes any molecule, e.g. protein or pharmaceutical, with the capability of modulating neurogenesis by acting through excitation pathways of neural progenitor cells. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally a plurality of assay mixtures are run in parallel with different agent concentrations to obtain a differential response to the various concentrations. Typically one of these concentrations serves as a negative control, i.e. at zero concentration or below the level of detection.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Compounds that are initially identified by any screening methods can be further tested to validate the apparent activity.

For identifying the mechanism of action and determining the cellular target an assay may contain specific and targeted alterations in the cell targeted for endoskeleton deposition, or functional modification of the endoskeleton. These alterations include addition or deletion of specific components, genetic alterations, or inclusion of specific compounds or interventions.

Various methods can be utilized for quantifying the presence of selected markers, for visualizing endoskeleton or other interacting cells, and the like. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. An abundance of useful dyes are now commercially available. These are available from many sources, including Sigma Chemical Company (St. Louis Mo.) and Molecular Probes (Handbook of Fluorescent Probes and Research Chemicals, Seventh Edition, Molecular Probes, Eugene Oreg.). Other fluorescent sensors have been designed to report on biological activities or environmental changes, e.g. pH, calcium concentration, electrical potential, proximity to other probes, etc. Methods of interest include calcium flux, nucleotide incorporation, quantitative PAGE (proteomics), etc.

Highly luminescent semiconductor quantum dots (zinc sulfide-capped cadmium selenide) have been covalently coupled to biomolecules for use in ultrasensitive biological detection (Stupp et al. (1997) Science 277(5330):1242-8; Chan et al. (1998) Science 281(5385):2016-8). Compared with conventional fluorophores, quantum dot nanocrystals have a narrow, tunable, symmetric emission spectrum and are photochemically stable (Bonadeo et al. (1998) Science 282(5393):1473-6). The advantage of quantum dots is the potential for exponentially large numbers of independent readouts from a single source or sample.

Multiple fluorescent labels can be used on the same sample and individually detected quantitatively, permitting measurement of multiple cellular responses simultaneously. Many quantitative techniques have been developed to harness the unique properties of fluorescence including: direct fluorescence measurements, fluorescence resonance energy transfer (FRET), fluorescence polarization or anisotropy (FP), time resolved fluorescence (TRF), fluorescence lifetime measurements (FLM), fluorescence correlation spectroscopy (FCS), and fluorescence photobleaching recovery (FPR) (Handbook of Fluorescent Probes and Research Chemicals, Seventh Edition, Molecular Probes, Eugene Oreg.).

Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target. U.S. Pat. No. 4,568,649 describes ligand detection systems, which employ scintillation counting. These techniques are particularly useful for protein or modified protein parameters or epitopes, or carbohydrate determinants. Readouts from such assays may be the mean fluorescence associated with individual fluorescent antibody-detected cell surface molecules or cytokines, or the average fluorescence intensity, the median fluorescence intensity, the variance in fluorescence intensity, or some relationship among these.

Identifiers of individual cells, for example different cell types or cell type variants, may be fluorescent, as for example labeling of different unit cell types with different levels of a fluorescent compound, and the like. If two cell types are to be mixed, one may be labeled and the other not. If three or more are to be included, each may be labeled to different levels of fluorescence by incubation with different concentrations of a labeling compound, or for different times. As identifiers of large numbers of cells, a matrix of fluorescence labeling intensities of two or more different fluorescent colors may be used, such that the number of distinct unit cell types that are identified is a number of fluorescent levels of one color, e.g., carboxyfluorescein succinimidyl ester (CFSE), times the number of fluorescence levels employed of the second color, e.g. tetramethylrhodamine isothiocyanate (TRITC), or the like, times the number of levels of a third color, etc. Alternatively, intrinsic light scattering properties of the different cell types, or characteristics of the biomaps of the test parameters included in the analysis, can be used in addition to or in place of fluorescent labels as unit cell type identifiers.

### Experimental

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Enduring Physical Structures Transmuted from Living Neural Circuitry

The ability to preserve the architecture of living brain tissue beyond its lifetime in vivo and in vitro in a targeted fashion can have broad implications for neuroscience research. We present a novel method, TEMPEST (Target-Element Modification by Physical and Enduring Structural Transmutation) to render specific networks in vivo durable and to easy visualize and manipulate them beyond the lifetime of the host.

We have previously developed and employed a method to control the activity of defined cell types in vivo with high temporal precision. To complement the functional control capabilities of optogenetics we are now introducing a method to preserve the structural integrity of defined brain circuits in vivo and in vitro. First, a series of polymers (chemical and biological) is optimized to fill and durabilize neurons. Next, the polymers are delivered in vivo in a cell-type specific manner. This circuit preservation method implemented with standard genetic tools to study any combination of intertwined nervous circuits, while maintaining their genetic identity. Circuits can be functionally addressed with optogenetics during behavioral paradigms, and then the relevant pathways can be durabilized and processed at later timepoints.

Durable materials from diverse sources could be used to create enduring neuronal tissue. Of particular interest are polymers that can be introduced genetically, to maintain the identity of the enduring cells, and that can fill thin neuronal process such as axons to preserve connectivity information. Such options can be enzyme-based polysaccharides (i.e. chitin, cellulose) or directly polymerizing non-neuronal proteins (i.e. silk, keratin). We therefore tested both chemical (chitin) and biological possibilities (keratin).

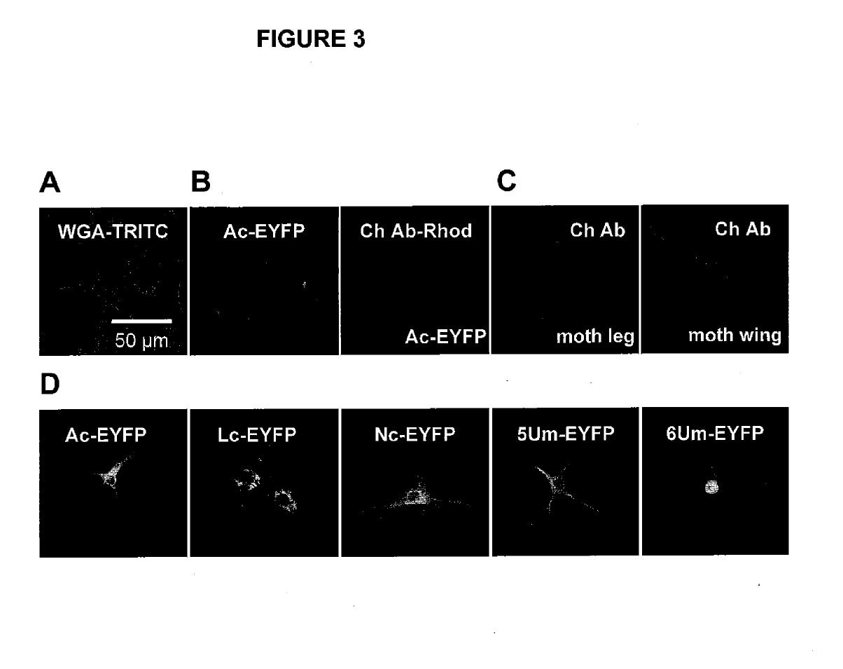

The strongest and most abundant material in nature is chitin, commonly building the walls of fungi and insects and protecting them from harsh conditions. Its strength and filamentous nature made it our first choice to test. Chitin is a polymer made of N-acetylglucosamine, which is also present in neurons (FIG. 3). Its synthesis is mediated by chitin synthase. In an attempt to synthesize chitin in mammalian cells we have expressed several chitin synthases from different organism in primary hippocampal neurons (FIG. 3D); Despite adding all necessary cofactors we failed to observe significant amounts of chitin (FIG. 3B,C). However, there is a possibility that the chitin gets secreted so further optimizations could achieve the goal.

Crossing evolutionary boundaries presents multiple challenges and we rationalized that a mammalian source would be more successful. A very strong candidate emerged in keratin, second only to chitin in strength, filamentous, of mammalian origin, and tremendous diversity (more than a dozen genes have been described). Keratin filaments are composed of two types of keratin: acidic and basic. Healthy epithelial cells produce keratin, then upon filling lose their nucleus and undergo programmed death.

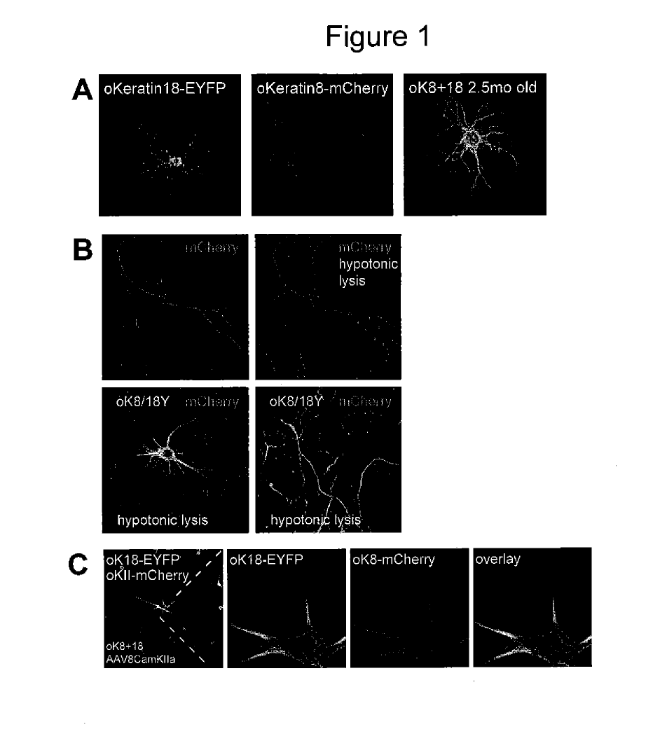

We synthesized multiple codon-optimized keratin pairs and fused them to fluorescent indicators. We then expressed the genes either alone (acidic or basic resulting in pepper-like expression) or in combination (resulting in nice long filaments filling the intracellular neuronal space) (FIG. 1A). The resulting keratin filaments (and therefore neuronal blueprint) were highly resistant to hypertonic lysis while the fluorescence only control quickly degraded (FIG. 1B). Even more, in transfected samples the keratin fluorescence can last for more than 4 months with cultures maintained untreated in the incubator while the regular fluorescence quickly fades (within a few weeks).

For cell-type specific targeting we made both lenti and adeno-associated viruses and infected cultured neurons. We used the CamKIIα promoter (previously published and tested) to express keratin only in excitatory neurons; keratin filaments were produced and filled neurites (FIG. 1C).

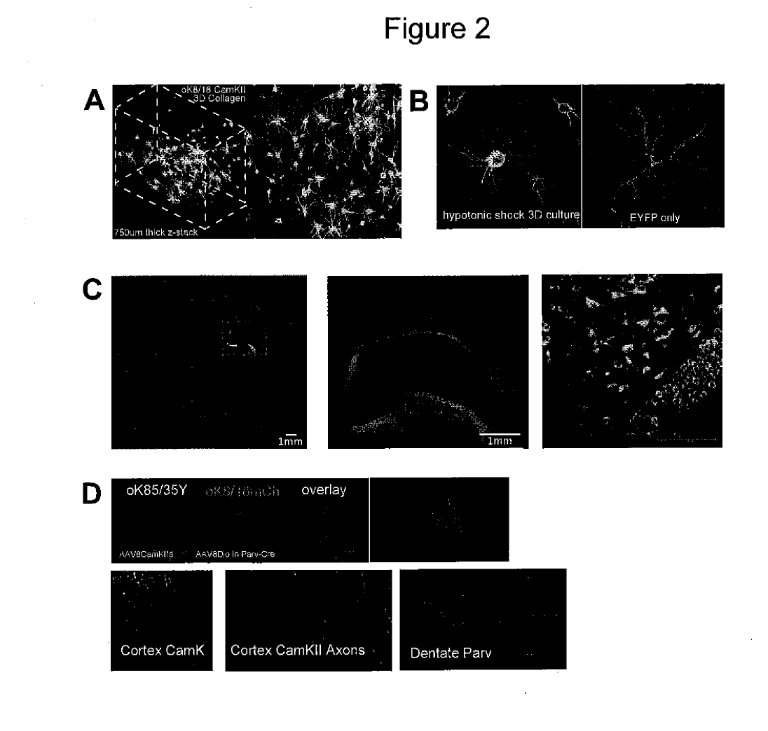

During the degradation process, although the keratinized neurons remain intact they lose support due to the disintegration of surrounding cells. To test durability of keratinized neurons against more harsh condition, we implemented a 3-D collagen culture and combined with viral transduction to obtain keratinized neurons in a supportive 3-D environment (FIG. 2A). The 3D cultured samples were then treated with proteases, detergent, and heat. Despite all the harsh treatments, the keratinized neurons were well preserved and maintain their shape and 3D arrangement while non-keratinized neurons quickly degraded (FIG. 2B).

Rationalizing that the brain can be seen as a big collagen block with intertwined circuits we attempted to endure defined neuronal circuits in vivo. We combined viral and transgenic approaches to express different pairs of keratins in either the excitatory (CamKIIα) or inhibitory (Parvalbumin) populations in cortex or hippocampus (FIG. 2D). Keratins are well expressed in vivo, fill processes and provide a durable, high-fidelity mask for the target cells (FIG. 2C).

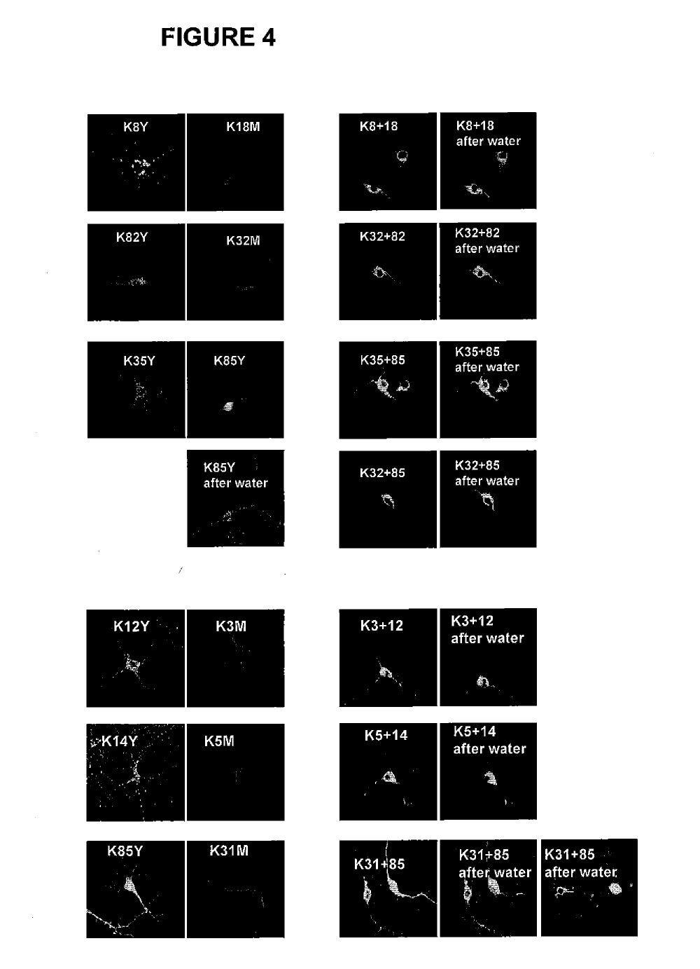

By taking advantage of the availability of antibodies against keratins the enduring networks could also be coated with materials of interest. In a proof-of-principle experiment we used a primary antibody to keratin followed by a colloidal gold conjugated secondary and then a gold enhancement strategy to grow the gold particles to connect with each other to form a continuous gold coating around the durable neuron. (FIG. 4).

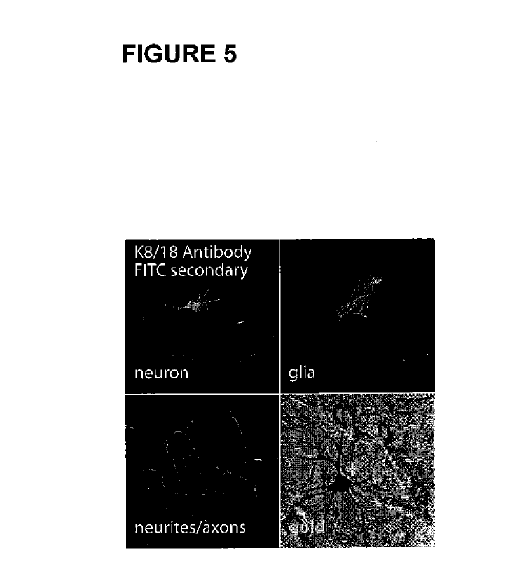

Because of the numerous keratin pairs available (FIG. 5), most of which have commercially available antibodies, multiple intermingled circuits can be imaged at the same time. If multiple circuits are targeted (more than the number of distinct fluorochromes available) the sample can be restrained and imaged multiple times and the circuits color-coded in software to obtain a cell-type specific rainbow.

We introduced TEMPEST (Target-Element Modification by Physical and Enduring Structural Transmutation), a method for creating durable structures in vivo in a cell-type and/or circuit specific manner via the use of insoluble polymers. TEMPEST provides is a way to functionally remove neurons while preserving their “shadow” for easy post-experiment detection and classification. With the appropriate choice of promoters or electroporation only a handful of cells could be removed and behavioral effects could be assessed. For example, under programmed cell-death markers, a durable polymer could be expressed via a strong acting virus (HSV, AAV-DJ) to remove that cell before immune response activation while still preserving its skeleton for later study (for example in PD or AD). Only a handful of cells could be lesioned this way and their loss-of-function assessed post behavioral studies; exactly what and how many cells were removed can be easily detected later on. Also, for big area lesions, the architecture is preserved so the tissue does not collapse. Future developments could expand TEMPEST to cover multiple classes of strong polymers (biological and chemical) and coating methods (drugs, small molecules, light-emitting, absorbing, reflective, or conductive materials) and expand the utility of the method.

### Methods

DNA constructs: All chitin synthases and keratin variants described here have been codon optimized for human and rodent expression and the optimized sequences were custom synthesized (DNA2.0, Inc., Menlo Park, Calif.).

All viral vectors were produced by PCR amplification and cloned in-frame into restriction sites of lentiviral or AAV vectors carrying different fluorochromes and the CaMKIIα or Synapsin-1 promoters according to standard molecular biology protocols. The lox-Cre strategy for expression in Cre mouse lines (Parvalbumin-Cre used here) has already been described elsewhere (Sohal et al., 2009; Tsai et al., 2009). All constructs were fully sequenced for accuracy of cloning; maps are available upon request.

Lentivirus preparation: Lentiviruses for cultured neuron infection and for in vivo injection were produced as previously described (Zhang et al., 2007b). The titer of viruses for culture infection was ˜10⁵ i.u./ml. The titer of concentrated virus for in vivo injection was ˜10¹⁰ i.u./ml.

Hippocampal cultures: Primary cultured hippocampal neurons were prepared from P0 Sprague-Dawley rat pups. The CA1 and CA3 regions were isolated, digested with 0.4 mg/mL papain (Worthington, Lakewood, N.J.), and plated onto glass coverslips precoated with 1:30 Matrigel (Beckton Dickinson Labware, Bedford, Mass.) at a density of 65,000/cm². Cultures were maintained in a 5% CO₂ humid incubator with Neurobasal-A medium (Invitrogen Carlsbad, Calif.) containing 1.25% FBS (Hyclone, Logan, Utah), 4% B-27 supplement (Gibco, Grand Island, N.Y.), 2 mM Glutamax (Gibco), and FUDR (2 mg/ml, Sigma, St. Louis, Mo.).

Calcium phosphate transfection: 6-10 div hippocampal neurons were grown at 65,000 cells/well in a 24-well plate. DNA/CaCl₂ mix for each well: 1.5-3 μg DNA (Qiagen endotoxin-free preparation)+1.875 μl 2M CaCl₂ (final Ca²⁺ concentration 250 mM) in 15 μI total H₂O. To DNA/CaCl₂ was added 15 μl of 2×HEPES-buffered saline (pH 7.05), and the final volume was mixed well by pipetting. After 20 min at RT, the 30 μl DNA/CaCl₂)₂/HBS mixture was dropped into each well (from which the growth medium had been temporarily removed and replaced with 400 μl warm MEM) and transfection allowed to proceed at 37 C for 45-60 minutes. Each well was then washed with 3×1 mL warm MEM and the growth medium replaced. Opsin expression was generally observed within 20-24 hours.

Immunohistochemistry: Primary hippocampal cultures were either transfected or infected with lentiviral or AAV8 virus (final dilution ˜10⁴ i.u./ml in neuronal growth medium). At 14 div cultures were fixed for 15 min with 4% paraformaldehyde and then permeabilized for 15 min with 0.1% triton X in 1% BSA and 2% normal goat serum (NGS). Primary antibody incubations were performed overnight at 4° C. using a antibodies against keratin (1:200). Alexa Fluor and Alexa Fluor Colloidal Gold-conjugated secondary antibodies (Invitrogen and Nanoprobes) were applied in 1% BSA and 2% NGS for 1 hour at room temperature. The colloidal gold secondary was followed by gold enhancement for bright filed visualization. Images were obtained on a confocal microscope using a dipping 25 X/0.95 NA water objective.

Stereotactic injection into the rodent brain: Adult mice, wild-type and Parv-Cre, were housed according to the approved protocols at Stanford. All surgeries were performed under aseptic conditions. The animals were anesthetized with anesthetic gas (isofluorane). The head was placed in a stereotactic apparatus (Kopf Instruments, Tujunga, Calif.; Olympus stereomicroscope). Ophthalmic ointment was applied to prevent eye drying. A midline scalp incision was made and a small craniotomy was performed using a drill mounted on the stereotactic apparatus (Fine Science Tools, Foster City, Calif.). The virus was delivered using a 10 μl syringe and a thin 34 gauge metal needle; the injection volume and flow rate (2 μl at 0.1 μI/min) was controlled with an injection pump from World Precision Instruments (Sarasota, Fla.). After injection the needle was left in place for 5 additional minutes and then slowly withdrawn. The skin was glued back with Vetbond tissue adhesive. The animal was kept on a heating pad until it recovered from anesthesia. Buprenorphine (0.03 mg/kg) was given subcutaneously following the surgical procedure to minimize discomfort. 2 μl of concentrated virus was microinjected at: anteroposterior −2 mm from bregma; lateral, −1 mm; ventral, 1.5 mm (For hippocampal expression); and AP, 0 mm from bregma; lateral, +1 mm; ventral, 1.0 mm) (for cortical expression). High-titer (2×10¹² g.c./mL) AAV8 was produced by the UNC VectorCore. For Parv-Cre injections, double-floxed cre-dependent AAV8 carrying the keratin genes was injected.

Tissue slice preparation: For preparation of brain slices, mice were sacrificed at various timepoints (1 week to 2 months) after viral injection. Rodents were perfused with 20 ml of ice-cold PBS, followed by 20 ml of fixative solution (2% paraformaldehyde; 2% monofixative). The brains were then fixed overnight in the fixative solution, and transferred to 30% sucrose solution for 2 days. Thick slices (>250 μm) were prepared using a Leica vibratome, and preserved in 4° C. in PBS. Slices (DAPI stain 1:50,000) were mounted with PVA-DABCO on microscope slides, and single confocal optical sections (e.g. through dorsal CA1 region, ˜1-2.5 mm posterior to bregma or the dorsal subiculum, 2.7-3 mm posterior to bregma) were acquired using a 10× air and 40 X/1.4 NA oil objectives on a Leica confocal microscope.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

1. A method of generating a stable endoskeleton in vivo with insoluble polymers, the method comprising:
targeting cells in an organ for expression of a genetic sequence that directly or indirectly gives rise to a stable endoskeleton structure within the cell; and
inducing expression of the genetic sequence to create the stable endoskeleton.

2. The method of claim 1, wherein the targeted cells are neurons.

3. The method of claim 2, wherein the neurons are CNS neurons.

4. the method of claim 1, wherein the targeted cells are present in an animal.

5. The method of claim 1, wherein the targeted cells are present in a tissue culture model.

6. The method of claim 1, further comprising the step of functionalizing the endoskeleton after deposition.

7. The method of claim 6, wherein the endoskeleton is functionalized for one or more of conduction of charge, conduction of drugs or fluids, conduction of growth factors or other elements, and the like.

8. The method of claim 1, wherein the endoskeleton is detectably labeled.

9. The method of claim 1, wherein the cell of interest is targeted by genetic, topologic, viral, structure, connectivity, promoters, tropisms, or other means.

10. The method of claim 1, wherein the genetic sequence directly gives rise to a stable endoskeleton.

11. The method of claim 10, wherein the genetic sequence encodes a polymer.

12. The method of claim 11, wherein the polymer is a keratin.

13. The method of claim 1, wherein the genetic sequence indirectly gives rise to an endoskeleton.

14. The method of claim 13, wherein the genetic sequence encodes enzymes that catalyze formation of an endoskeleton from monomers normally present or provided to the cell.

15. The method of claim 1, further comprising the step of removing the organ structure around the endoskeleton.

16. The method of claim 15, wherein the endoskeleton is provided with three-dimensional support.

17. The method according to claim 1, wherein two or more different endoskeletons are induced in the organ.

18. The method according to claim 1, comprising the step of analyzing the remaining non-modified cells may be studied for function, gene expression, behavior, electrochemistry, and the like, to determine the effect of selective inactivation of the targeted cells.

19. The method according to claim 1, further comprising the step of applying a candidate treatment or agent to the organ before, during or after endoskeleton deposition to determine the effect of the treatment agent on cells in the absence or presence of the targeted cells.

20. The method according to claim 1, further comprising studying the resulting physical structure for its physical connectivity, mapped functionally with regard to dynamics and circuit flow, as a source of fundamental insight into cellular circuit function, a means of mapping and understanding circuit pathologies, a technique for screening and identifying interventions to correct circuit abnormalities, a means of permanently storing or immortalizing cellular circuits in terms of structure, connectivity, identity and functionality, and a technique for extending or expanding brain function, human or otherwise, in terms of capacity, complexity, consciousness, or power.
